(19) United States
(12) Patent Application Publication
(10) Pub. No.: US 2023/0306605 A1
(43) Pub. Date: Sep. 28, 2023

(54) IMAGE GENERATION APPARATUS, METHOD, AND PROGRAM, LEARNING APPARATUS, METHOD, AND PROGRAM, SEGMENTATION MODEL, AND IMAGE PROCESSING APPARATUS, METHOD, AND PROGRAM

(71) Applicant: FUJIFILM Corporation, Tokyo (JP)

(72) Inventors: Saeko SASUGA, Tokyo (JP); Yoshiro Kitamura, Tokyo (JP); Akira Kudo, Tokyo (JP)

(73) Assignee: FUJIFILM Corporation, Tokyo (JP)

(21) Appl. No.: 18/170,532

(22) Filed: Feb. 17, 2023

(30) Foreign Application Priority Data
Mar. 25, 2022 (JP) 2022-050635
Sep. 21, 2022 (JP) 2022-150250

(51) Int. Cl. G06T 7/11; G06T 11/00; G06T 7/174; G06T 7/00; G16H 30/40

(52) U.S. Cl. CPC G06T 7/11; G06T 11/00; G06T 7/174; G06T 7/0012; G16H 30/40; G06T 2207/30008; G06T 2207/30096; G06T 2207/20081

(57) ABSTRACT

A processor is configured to acquire an original image and a mask image in which masks are applied to one or more regions respectively representing one or more objects including a target object in the original image, derive a pseudo mask image by processing the mask in the mask image, and derive a pseudo image that has a region based on a mask included in the pseudo mask image and has the same representation format as the original image, based on the original image and the pseudo mask image.

### CROSS REFERENCE TO RELATED APPLICATIONS

The present application claims priority from Japanese Patent Application No. 2022-050635, filed on Mar. 25, 2022 and Japanese Patent Application No. 2022-150250, filed on Sep. 21, 2022, the entire disclosures of which are incorporated herein by reference.

### BACKGROUND

### Technical Field

The present disclosure relates to an image generation apparatus, a method, and a program, a learning apparatus, a method, and a program, a segmentation model, and an image processing apparatus, a method, and a program.

### Related Art

As a machine learning model that handles images, a convolutional neural network (hereinafter, abbreviated as CNN) that performs semantic segmentation for identifying a target object included in an image in units of pixels is known. For example, U-Net: Convolutional Networks for Biomedical Image Segmentation, Olaf Ronneberger, et al., 2015 has suggested segmentation using a U-shaped convolutional neural network (U-Net; U-Shaped Neural Network).

In the medical field, a medical image is segmented using a machine learning model, and determination of a progress of an illness on a segmented region is performed.

On the other hand, a lot of training data is required for learning of the machine learning model. Note that, in the medical field, since collection of data of a scarce disease is difficult, it is difficult to provide a machine learning model capable of accurately performing segmentation.

For this reason, for learning of a machine learning model for detecting a skin cancer, a technique that generates a pseudo image including skin cancers of various sizes using an existing medical image and correct answer data in which a region of a skin cancer in the medical image is masked has been suggested (see Mask2Lesion: Mask-Constrained Adversarial Skin Lesion Image Synthesis, Kumar Abhishek, et al., 2019). For example, a technique that learns a spatial distribution of a three-dimensional object having an existing shape, such as a chair, and generates an image of a chair having an unknown shape has also been suggested (see The shape variational autoencoder: A deep generative model of part-segmented 3D objects, C. Nash, et al., 2017).

Note that, like the technique described in Mask2Lesion: Mask-Constrained Adversarial Skin Lesion Image Synthesis, Kumar Abhishek, et al., 2019, it is not possible to generate data of features that are rarely included in existing learning data, only using correct answer data on an existing image. For this reason, it is difficult to construct a machine learning model capable of accurately segmenting a target object that is rarely included in an existing image, even though a generated image is added to learning data. Like the technique described in The shape variational autoencoder: A deep generative model of part-segmented 3D objects, C. Nash, et al., 2017, it is difficult to construct a machine learning model capable of accurately segmenting a target object different from an existing target object only by changing a shape of the existing target object. In particular, on a scarce disease, such as a progressive cancer, since a cancer tissue is often infiltrated into a part in the vicinity thereof, there is a demand for segmenting such a progressive cancer with excellent accuracy.

### SUMMARY OF THE INVENTION

The present disclosure has been accomplished in view of the above-described situation, and an object of the present disclosure is to provide a machine learning model capable of accurately performing segmentation.

An image generation apparatus according to a first aspect of the present disclosure comprises at least one processor, in which the processor is configured to acquire an original image and a mask image in which masks are applied to one or more regions respectively representing one or more objects including a target object in the original image, derive a pseudo mask image by processing the mask in the mask image, and derive a pseudo image that has a region based on a mask included in the pseudo mask image and has the same representation format as the original image, based on the original image and the pseudo mask image.

An image generation apparatus according to a second aspect of the present disclosure is the image generation apparatus according to the first aspect of the present disclosure, in which the pseudo mask image and the pseudo image may be used as training data for learning a segmentation model that segments the object included in an image.

An image generation apparatus according to a third aspect of the present disclosure is the image generation apparatus according to the second aspect of the present disclosure, in which the processor may be configured to accumulate the pseudo mask image and the pseudo image as training data.

An image generation apparatus according to a fourth aspect of the present disclosure is the image generation apparatus according to any one of the first to third aspects of the present disclosure, in which the processor may be configured to derive the pseudo mask image that is able to generate the pseudo image including a target object of a class different from a class indicated by the target object.

The term “class is different” means that a type of a shape of the target object is different, that, in a case where the target object is a lesion included in a medical image, a progress of the lesion is different. The term “different class” means a class that has a small frequency of appearance or is not at all compared to other classes, in stored training data. For this reason, “the pseudo mask image that is able to generate the pseudo image including the target object of the class different from the class indicated by the target object” is derived, whereby it is possible to prepare training data of a class that is small or is not at all in existing training data. Accordingly, learning of the segmentation model is performed using such training data along with existing training data, whereby it is possible to construct a segmentation model that can segment a target object on an image including a target object having a small frequency of appearance.

An image generation apparatus according to a fifth aspect of the present disclosure is the image generation apparatus according to any one of the first to fourth aspects of the present disclosure, in which the processor may be configured to derive the pseudo mask image by processing the mask such that at least one of a shape or a progress of a lesion is different from that of a lesion included in the original image, based on a lesion shape evaluation index used as an evaluation index in medical practice for a medical image.

An image generation apparatus according to a sixth aspect of the present disclosure is the image generation apparatus according to any one of the first to fifth aspects of the present disclosure, in which the processor may be configured to derive the pseudo mask image by processing the mask until a normal organ has a shape to be evaluated as a lesion based on a measurement index in medical practice for a medical image.

An image generation apparatus according to a seventh aspect of the present disclosure is the image generation apparatus according to any one of the first to sixth aspects of the present disclosure, in which the processor may be configured to refer to at least one style image having predetermined density, color, or texture and generate the pseudo image having density, color, or texture depending on the style image.

The term “style image” is an image that represents an object of the same type as the target object having possible density, color, and texture of the target object.

An image generation apparatus according to an eighth aspect of the present disclosure is the image generation apparatus according to any one of the first to seventh aspects of the present disclosure, in which the processor may be configured to receive an instruction for a degree of processing of the mask and derive the pseudo mask image by processing the mask based on the instruction.

An image generation apparatus according to a ninth aspect of the present disclosure is the image generation apparatus according to the eighth aspect of the present disclosure, in which the processor may be configured to receive designation of a position of an end point of the mask after processing and designation of a processing amount as the instruction for the degree of processing.

An image generation apparatus according to a tenth aspect of the present disclosure is the image generation apparatus according to the eighth or ninth aspect of the present disclosure, in which the processor may be configured to receive the instruction for the degree of processing of the mask under a constraint condition set in advance.

An image generation apparatus according to an eleventh aspect of the present disclosure is the image generation apparatus according to any one of the first to tenth aspects of the present disclosure, in which, in a case where the original image includes a plurality of the objects, and the target object and a partial region of another object other than the target object have an inclusion relation, in the mask image, a region having the inclusion relation may be applied with a mask different from a region having no inclusion relation.

An image generation apparatus according to a twelfth aspect of the present disclosure is the image generation apparatus according to the eleventh aspect of the present disclosure, in which the processor may be configured to, in a case where the other object having the inclusion relation is an object fixed in the original image, derive the pseudo mask image by processing the mask applied to the target object conforming to a shape of a mask applied to the fixed object.

An image generation apparatus according to a thirteenth aspect of the present disclosure is the image generation apparatus according to any one of the first to twelfth aspects of the present disclosure, in which the processor may be configured to, in a case where the original image is a three-dimensional image, derive the pseudo mask image by processing the mask while maintaining three-dimensional continuity of the mask applied to the region of the target object.

An image generation apparatus according to a fourteenth aspect of the present disclosure is the image generation apparatus according to any one of the first to thirteenth aspects of the present disclosure, in which the original image may be a three-dimensional medical image, and the target object may be a lesion included in the medical image.

An image generation apparatus according to a fifteenth aspect of the present disclosure is the image generation apparatus according to the fourteenth aspect of the present disclosure, in which the medical image may include a rectum of a human body, and the target object may be a rectal cancer, and another object other than the target object may be at least one of a mucous membrane layer of the rectum, a submucosal layer of the rectum, a muscularis propria of the rectum, a subserous layer of the rectum, or a background other than the layers.

An image generation apparatus according to a sixteenth aspect of the present disclosure is the image generation apparatus according to the fourteenth aspect of the present disclosure, in which the medical image may include a joint of a human body, and the target object may be a bone composing the joint, and another object other than the target object may be a background other than the bone composing the joint.

A learning apparatus according to a seventeenth aspect of the present disclosure comprises at least one processor, in which the processor is configured to construct a segmentation model that segments a region of one or more objects including a target object included in an input image, by performing machine learning using a plurality of sets of pseudo images and pseudo mask images generated by the image generation apparatus according to any one of the first to sixteenth aspects of the present disclosure as training data.

A learning apparatus according to an eighteenth aspect of the present disclosure is the learning apparatus according to the seventeenth aspect of the present disclosure, in which the processor may be configured to construct the segmentation model by performing machine learning using a plurality of sets of original images and mask images as training data.

A segmentation model according to a nineteenth aspect of the present disclosure is constructed by the learning apparatus according to the seventeenth or eighteenth aspect of the present disclosure.

An image processing apparatus according to a twentieth aspect of the present disclosure comprises at least one processor, in which the processor is configured to derive a mask image in which one or more objects included in a target image to be processed are masked, by segmenting a region of one or more objects including a target object included in the target image using the segmentation model according to the nineteenth aspect of the present disclosure.

An image processing apparatus according to the twenty-first aspect of the present disclosure, in the image processing apparatus according to the twentieth aspect of the present disclosure, in which the processor may be configured to discriminate a class of the target object masked in the mask image using a discrimination model that discriminates a class of a target object included in a mask image.

An image generation method according to a twenty-second aspect of the present disclosure comprises acquiring an original image and a mask image in which masks are applied to one or more regions respectively representing one or more objects including a target object in the original image, deriving a pseudo mask image by processing the mask in the mask image, and deriving a pseudo image that has a region based on a mask included in the pseudo mask image and has the same representation format as the original image, based on the original image and the pseudo mask image.

A learning method according to a twenty-third aspect of the present disclosure constructs a segmentation model that segments a region of one or more objects including a target object included in an input image, by performing machine learning using a plurality of sets of pseudo images and pseudo mask images generated by the image generation method according to the twenty-second aspect of the present disclosure as training data.

An image processing method according to a twenty-fourth aspect of the present disclosure comprises deriving a mask image in which one or more objects included in a target image to be processed are masked, by segmenting a region of one or more objects including a target object included in the target image using the segmentation model according to the nineteenth aspect of the present disclosure.

An image generation program according to a twenty-fifth aspect of the present disclosure causes a computer to execute procedure of acquiring an original image and a mask image in which masks are applied to one or more regions respectively representing one or more objects including a target object in the original image, a procedure of deriving a pseudo mask image by processing the mask in the mask image, and a procedure of deriving a pseudo image that has a region based on a mask included in the pseudo mask image and has the same representation format as the original image, based on the original image and the pseudo mask image.

A learning program according to a twenty-sixth aspect of the present disclosure causes a computer to execute a procedure of constructing a segmentation model that segments a region of one or more objects including a target object included in an input image, by performing machine learning using a plurality of sets of pseudo images and pseudo mask images generated by the image generation method according to the twenty-second aspect of the present disclosure as training data.

An image processing program according to a twenty-aspect of the present disclosure causes a computer to execute a procedure of deriving a mask image in which one or more objects included in a target image to be processed are masked, by segmenting a region of one or more objects including a target object included in the target image using the segmentation model according to the nineteenth aspect of the present disclosure.

According to the present disclosure, it is possible to provide a machine learning model capable of accurately segmentation.

### BRIEF DESCRIPTION OF THE DRAWINGS

FIG. 1 is a diagram showing the schematic configuration of a medical information system to which an image generation apparatus, a learning apparatus, and an image processing apparatus according to an embodiment of the present disclosure are applied.

FIG. 2 is a diagram showing the hardware configuration of the image generation apparatus and the learning apparatus according to the present embodiment.

FIG. 3 is a functional configuration diagram of the image generation apparatus and the learning apparatus according to the present embodiment.

FIG. 4 is a diagram schematically showing a cross section of a rectum for describing progress of a rectal cancer.

FIG. 5 is a diagram showing an example of an original image and a mask image.

FIG. 6 is a diagram showing a mask processing screen on a rectal cancer.

FIG. 7 is a diagram showing a mask processing screen on the rectal cancer.

FIG. 8 is a diagram showing a mask processing screen on the rectal cancer.

FIG. 9 is a diagram showing another example of mask processing.

FIG. 10 is a diagram showing another example of mask processing.

FIG. 11 is a diagram schematically showing a generator and learning thereof.

FIG. 12 is a diagram showing a pseudo mask and a pseudo image.

FIG. 13 is a diagram showing the hardware configuration of the image processing apparatus according to the present embodiment.

FIG. 14 is a functional configuration diagram of the image processing apparatus according to the present embodiment.

FIG. 15 is a diagram showing a display screen.

FIG. 16 is a flowchart illustrating image generation processing in the present embodiment.

FIG. 17 is a flowchart illustrating learning processing in the present embodiment.

FIG. 18 is a flowchart illustrating image processing in the present embodiment.

FIG. 19 is a diagram showing a mask processing screen on a bone spur.

FIG. 20 is a diagram showing a mask processing screen on the bone spur.

FIG. 21 is a diagram showing another example of a mask processing screen.

FIG. 22 is a diagram showing another example of a mask processing screen.

FIG. 23 is a diagram showing another example of a mask processing screen.

FIG. 24 is a diagram showing another example of a mask processing screen.

FIG. 25 is a diagram showing another example of a mask processing screen.

FIG. 26 is a diagram showing another example of a mask processing screen.

FIG. 27 is a diagram showing another example of a mask processing screen.

FIG. 28 is a diagram showing another example of a mask processing screen.

FIG. 29 is a diagram showing another example of a mask processing screen.

FIG. 30 is a diagram showing another example of a mask processing screen.

### DETAILED DESCRIPTION

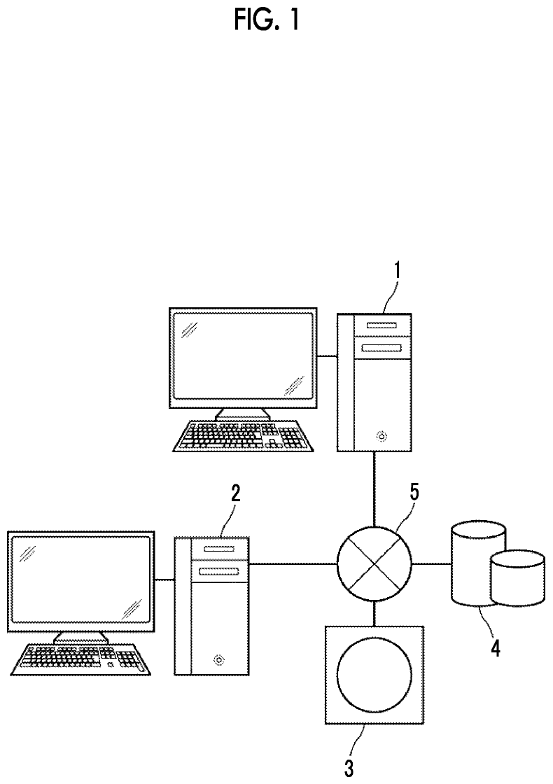

Hereinafter, an embodiment of the present disclosure will be described referring to the drawings. First, the configuration of a medical information system to which an image generation apparatus, a learning apparatus, and an image processing apparatus according to the present embodiment are applied will be described. FIG. 1 is a diagram showing the schematic configuration of the medical information system. The medical information system shown in FIG. 1 has a configuration in which a computer 1 including the image generation apparatus and the learning apparatus according to the present embodiment, a computer 2 including the image processing apparatus according to the present embodiment, an imaging apparatus 3, and an image storage server 4 are connected in a communicable state by way of a network 5.

The computer 1 includes the image generation apparatus and the learning apparatus according to the present embodiment, and an image generation program and a learning program according to the present embodiment are installed thereon. The computer 1 may be a workstation or a personal computer or may be a server computer connected to the workstation or the personal computer through a network. The image generation program and the learning program are stored in a storage device of the server computer connected to the network or a network storage in a state of being accessible from the outside, and are downloaded to and installed on the computer 1 on demand. The image generation program and the learning program are recorded on a recording medium, such as a digital versatile disc (DVD) or a compact disc read only memory (CD-ROM), are distributed, and are installed on the computer 1 from the recording medium.

The computer 2 includes the image processing apparatus according to the present embodiment, and the image processing program of the present embodiment is installed thereon. The computer 2 may be a workstation or a personal computer or may be a server computer connected to the workstation or the personal computer through a network. The image processing program is stored in a storage device of the server computer connected to the network or a network storage in a state of being accessible from the outside, and is downloaded to and installed on the computer 2 on demand. The image processing program is recorded on a recording medium, such as a DVD or a CD-ROM, is distributed, and is installed on the computer 2 from the recording medium.

The imaging apparatus 3 is an apparatus that images a part to be diagnosed of a subject to generate a three-dimensional image representing the part, and specifically, is a CT apparatus, an MM apparatus, a positron emission tomography (PET) apparatus, or the like. The three-dimensional image generated by the imaging apparatus 3 and composed of a plurality of tomographic images is transmitted to and stored in the image storage server 4. In the present embodiment, the imaging apparatus 3 is an MRI apparatus, and generates an MRI image of a human body as the subject as a three-dimensional image. In the present embodiment, it is assumed that the three-dimensional image is a three-dimensional image including the vicinity of a rectum of the human body. For this reason, in a case where a patient with a rectal cancer is imaged, the rectal cancer is included in the three-dimensional image.

The image storage server 4 is a computer that stores and manages various kinds of data, and comprises a large capacity external storage device and software for database management. The image storage server 4 performs communication with other apparatuses through the network 5 in a wired or wireless manner and transmits and receives image data and the like. Specifically, the image storage server 4 acquires various kinds of data including image data of the three-dimensional image generated by the imaging apparatus 3 by way of the network, and stores and manages the acquired data in a recording medium, such as a large capacity external storage device. In the image storage server 4, training data for constructing a machine learning model for deriving a pseudo image, detecting an abnormal part, or discriminating a class of the abnormal part as described below is also stored. A storage format of image data and communication between the respective apparatuses by way of the network 5 are based on a protocol, such as Digital Imaging and Communication in Medicine (DICOM).

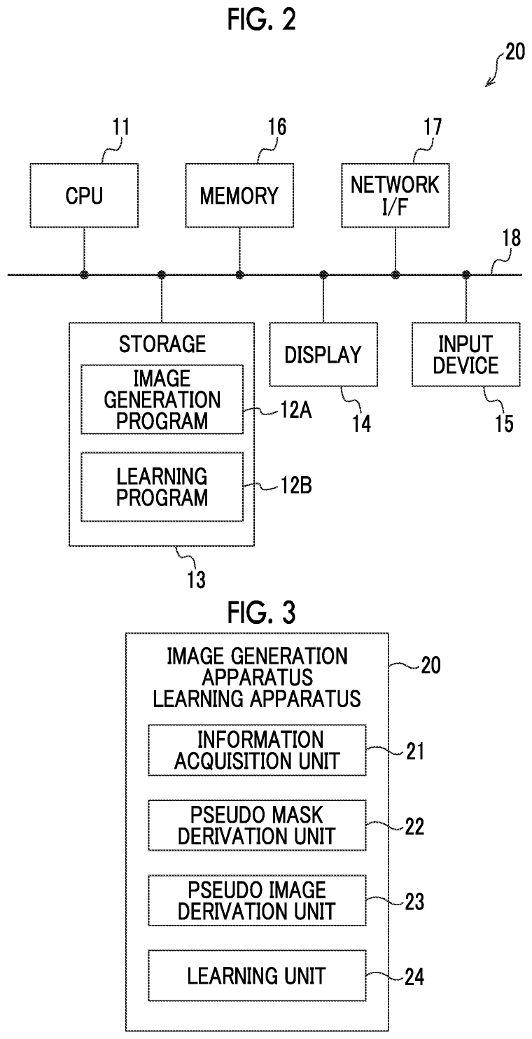

Next, the image generation apparatus and the learning apparatus according to the present embodiment will be described. FIG. 2 is a diagram showing the hardware configuration of the image generation apparatus and the learning apparatus according to the present embodiment. As shown in FIG. 2, the image generation apparatus and the learning apparatus (hereinafter, represented by the image generation apparatus) 20 include a central processing unit (CPU) 11, a non-volatile storage 13, and a memory 16 as a temporary storage region. The image generation apparatus 20 includes a display 14, such as a liquid crystal display, an input device 15, such as a keyboard and a mouse, and a network interface (I/F) 17 that is connected to the network 5. The CPU 11, the storage 13, the display 14, the input device 15, the memory 16, and the network I/F 17 are connected to a bus 18. The CPU 11 is an example of a processor in the present disclosure.

The storage 13 is realized by a hard disk drive (HDD), a solid state drive (SSD), a flash memory, or the like. In the storage 13 as a storage medium, an image generation program 12A and a learning program 12B are stored. The CPU 11 reads out the image generation program 12A and the learning program 12B from the storage 13, develops the image generation program 12A and the learning program 12B to the memory 16, and executes the developed image generation program 12A and learning program 12B.

Next, the functional configuration of the image generation apparatus and the learning apparatus according to the present embodiment will be described. FIG. 3 is a diagram showing the functional configuration of the image generation apparatus and the learning apparatus according to the present embodiment. As shown in FIG. 3, the image generation apparatus 20 comprises an information acquisition unit 21, a pseudo mask derivation unit 22, a pseudo image derivation unit 23, and a learning unit 24. Then, the CPU 11 executes the image generation program 12A, whereby the CPU 11 functions as the information acquisition unit 21, the pseudo mask derivation unit 22, and the pseudo image derivation unit 23. The CPU executes the learning program 12B, whereby the CPU 11 functions as the learning unit 24.

The information acquisition unit 21 acquires an original image G0 that is used to derive a pseudo image described below, from the image storage server 4. The information acquisition unit 21 acquires training data for constructing a trained model described below from the image storage server 4.

Here, in the present embodiment, the original image G0 is stored in conjunction with a mask image M0 in which a mask is applied to a region of an object included in the original image G0. In a case where a rectal cancer is included in the original image G0, information representing a stage of the rectal cancer is applied to the original image G0.

The application of the mask may be performed by a manual operation using the input device 15 to the original image G0 or may be performed by segmenting the original image G0. As the segmentation, semantic segmentation that performs class classification by labeling all pixels of an image in units of pixels is used. The semantic segmentation is performed by a semantic segmentation model that is a machine learning model constructed by performing machine learning to extract a region of an object included in an image. The semantic segmentation model will be described below. The original image G0 and the mask image M0 that are stored in the image storage server 4 may be images derived by the image processing apparatus of the present embodiment described below.

In the present embodiment, a rectal cancer is detected from a target image to be processed in the image processing apparatus described below. For this reason, a mask for identifying each region of the rectal cancer included in the original image G0, a mucous membrane layer of a rectum, a submucosal layer of the rectum, a muscularis propria of the rectum, a subserous layer of the rectum, and a background other than the layers is applied as the mask image M0 to the original image G0. The rectal cancer included in the original image G0 is an example of a target object, and the mucous membrane layer of the rectum, the submucosal layer of the rectum, the muscularis propria of the rectum, the subserous layer of the rectum, and the background other than the layers are an example of objects other than the target object.

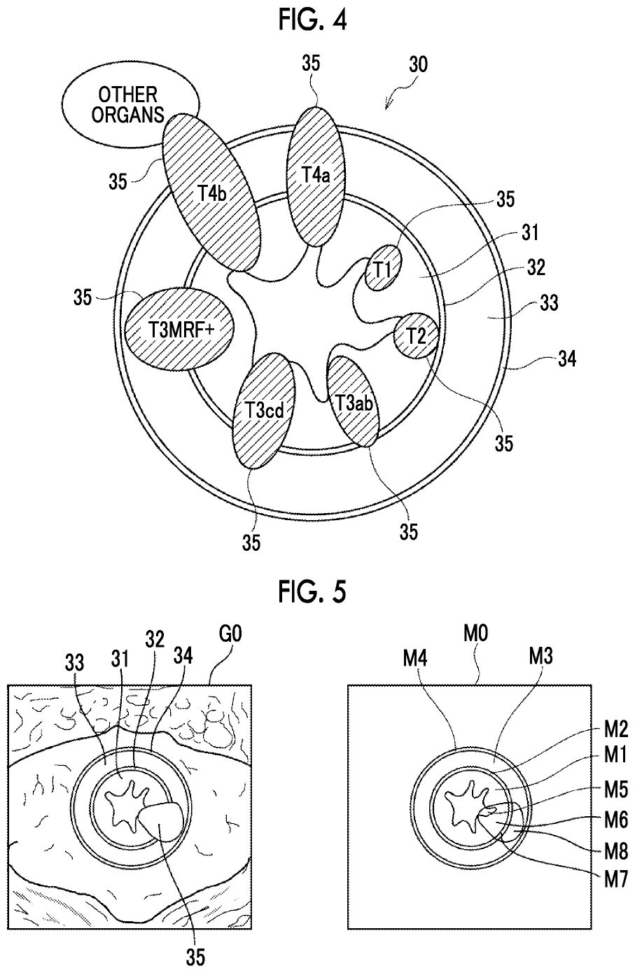

Here, while an initial rectal cancer is present only in the mucous membrane layer, in a case where the rectal cancer progresses, the rectal cancer spreads outward from the mucous membrane layer, is infiltrated into the submucosal layer and the muscularis propria, and has an inclusion relation with the submucosal layer and the muscularis propria. FIG. 4 is a diagram schematically showing a cross section of a rectum for describing the progress of the rectal cancer. As shown in FIG. 4, a rectum 30 is composed of a mucous membrane layer 31, a submucosal layer 32, a muscularis propria 33, and a subserous layer 34. In FIG. 4, regions of a rectal cancer 35 depending on a degree of cancer progress, that is, a cancer stage are hatched. As shown in FIG. 4, while the rectal cancer 35 of initial stages T1 and T2 is positioned in the mucous membrane layer 31 of the rectum 30, in a case where the rectal cancer 35 progresses, the rectal cancer 35 is infiltrated into the submucosal layer 32 (stage T3ab), is further infiltrated into the muscularis propria 33 (stage T3cd), and gradually approaches the subserous layer 34 (stage T3MRF+). In a case where the rectal cancer further progresses, the rectal cancer breaks through the muscularis propria 33 and the subserous layer 34 (stage T4a), and reaches other organs (stage T4b). The respective stages of the rectal cancer correspond to different classes of the present disclosure.

FIG. 5 is a diagram showing examples of the original image G0 and the mask image M0. The original image G0 shown in FIG. 5 shows a tomographic plane of a portion in a rectum where a rectal cancer is present, taken in a direction crossing a central axis of the rectum. In the original image G0 shown in FIG. 5, a rectal cancer 35 spreads outward from the mucous membrane layer 31, is infiltrated into the submucosal layer 32 and the muscularis propria 33, and as a result, the rectal cancer 35, and the mucous membrane layer 31, the submucosal layer 32, and the muscularis propria 33 have an inclusion relation. In FIG. 5, a rectal cancer 35 is not infiltrated into the subserous layer 34.

In the present embodiment, a region having an inclusion relation in the original image G0 is applied with a mask different from a region having no inclusion relation. For example, in the mask image M0 shown in FIG. 5, masks M1, M2, M3, and M4 are applied to the mucous membrane layer 31, the submucosal layer 32, the muscularis propria 33, and the subserous layer 34, respectively. A mask M5 is applied to a region in the rectal cancer 35 having no inclusion relation with any tissue of the rectum. In the rectal cancer 35, a mask M6 is applied to a region having an inclusion relation with the mucous membrane layer 31, a mask M7 is applied to a region having an inclusion relation with the submucosal layer 32, and a mask M8 is applied to a region having an inclusion relation with the muscularis propria 33. In FIG. 5, a mask of the background is omitted.

In a case of deriving the mask image M0 by the segmentation model, to segment a region having an inclusion relation by the semantic segmentation model, the segmentation model may be constructed by performing machine learning while preparing training data including correct answer data with a region having an inclusion relation and a region having no inclusion relation applied with different masks.

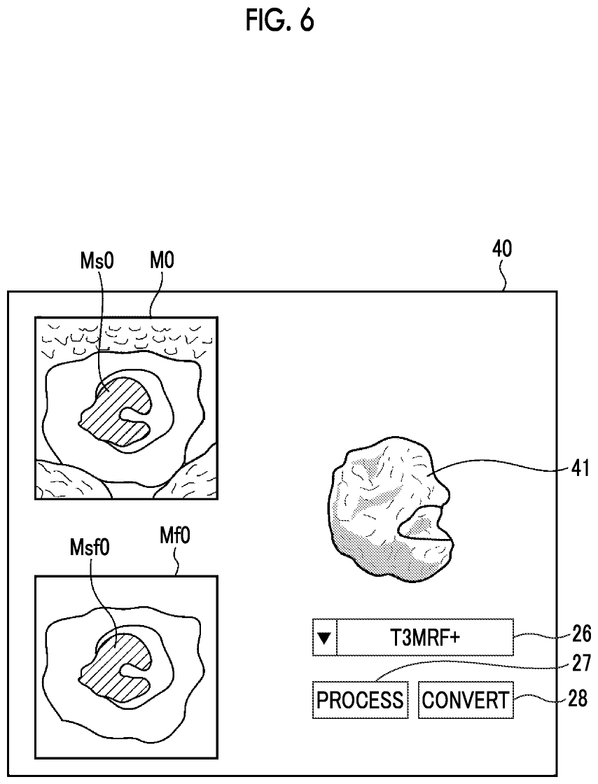

The pseudo mask derivation unit 22 derives a pseudo mask image by processing the masks included in the mask image M0 of the original image G0. To this end, a display control unit 74 displays a mask processing screen on the display 14. FIG. 6 is a diagram showing a mask processing screen. As shown in FIG. 6, on a mask processing screen 40, the mask image M0, a pseudo mask image Mf0 including a mask Mf0 obtained by processing the mask M0 included in the mask image M0, a three-dimensional model 41 of the rectal cancer included in the original image G0, a pull-down menu 26 for setting a degree of deformation of the three-dimensional model 41, a PROCESS button 27 for executing mask processing, and a CONVERT button 28 for deriving a pseudo image are displayed.

The mask image M0 and the pseudo mask image Mf0 shown in FIG. 6 show a tomographic plane of a portion in the rectum where the rectal cancer is present, taken in a direction crossing a central axis of a rectum. Here, for description, the mask image M0 shown in FIG. 6 is an image in which a mask Ms0 is applied to only a region of a rectal cancer to be processed in the original image G0. In the pseudo mask image Mf0, only a mask of the rectal cancer to be processed is represented by a reference numeral Msf0. The mask image M0 and the pseudo mask image Mf0 shown in FIG. 6 are images before mask processing, the masks Ms0 and Msf0 applied to the respective images have the same shape. Here, the rectal cancer show in FIG. 6 is in the stage T3ab as the stage of the rectal cancer shown in FIG. 4.

The three-dimensional model 41 is a three-dimensional image derived by extracting only the region of the rectal cancer in the original image G0 and performing volume rendering. An operator can change omnidirectional orientations of the three-dimensional model 41 by operating the input device 15.

In the pull-down menu 26, the stage of the rectal cancer can be selected. That is, in the pull-down menu 26, the stages T1, T2, T3ab, T3cd, T3MRF+, T4a, and T4b of the rectal cancer shown in FIG. 4 can be selected.

In the present embodiment, the pseudo mask derivation unit 22 derives a pseudo mask image capable of generating a pseudo image including a target object of a class different from the class of the target object. That is, in deriving the pseudo mask image Mf0, the pseudo mask derivation unit 22 processes a mask while deforming the three-dimensional model 41 such that a pseudo image including a rectal cancer of a stage different from the stage of the rectal cancer included in the original image G0 can be generated. For example, in a case where the stage of the rectal cancer included in the original image G0 is the stage T1 where the rectal cancer is present only in the mucous membrane layer, the three-dimensional model 41 corresponds to, for example, the rectal cancer of the stage T3ab progressed from the stage T1 by deforming the three-dimensional model 41 of the rectal cancer to extend to the muscularis propria. The three-dimensional model 41 corresponds to the rectal cancer of the stage T4a by deforming the three-dimensional model 41 of the rectal cancer to break through the muscularis propria.

For this reason, the operator views the displayed mask image M0 and selects a stage of a rectal cancer to be generated as a pseudo image from the pull-down menu 26. FIG. 6 shows a state in which the stage T3MRF+ is selected. After the selection of the stage of the rectal cancer, in a case where the PROCESS button 27 is selected by the operator, the pseudo mask derivation unit 22 deforms the three-dimensional model 41 such that the stage of the rectal cancer represented by the three-dimensional model 41 is T3MRF+. The pseudo mask derivation unit 22 processes the mask Msf0 applied to the pseudo mask image Mf0 to conform to the shape of the deformed three-dimensional model 41.

In deriving the pseudo mask image Mf0, the pseudo mask derivation unit 22 deforms the three-dimensional model 41 such that a shape and/or a progress of a lesion is different from that of a lesion included in the original image G0 based on a lesion shape evaluation index used as an evaluation index in medical practice for a medical image. That is, the shape of the rectal cancer is deformed such that the shape and/or the progress of the rectal cancer included in the original image G0 is turned from the stage T3ab to the stage T3MRF+, for example. Alternatively, the three-dimensional model 41 is deformed until a normal organ has a shape to be evaluated as a lesion based on a measurement index in medical practice for a medical image.

The pseudo mask derivation unit 22 processes the mask while maintaining the three-dimensional continuity of the mask applied to the rectal cancer. For example, in deforming the three-dimensional model 41 to extend toward the outside of the rectum, a degree of deformation decreases as far from the center of the rectum in the three-dimensional model 41. With this, it is possible to deform the three-dimensional model 41 while maintaining the three-dimensional continuity in the original three-dimensional model 41, and as a result, it is possible to process the mask while maintaining the three-dimensional continuity of the mask applied to the rectal cancer.

The three-dimensional model 41 corresponds to the rectal cancer, and deforming the three-dimensional model 41 to make the stage of the rectal cancer progress is making the rectal cancer be infiltrated into the submucosal layer and the muscularis propria of the rectum. Here, while the rectal cancer is enlarged or deformed with the progress, a way of deformation depends on the shape of the rectum. That is, the rectal cancer is enlarged or deformed to match the shape of the rectum. Here, the rectum is fixed in the original image G0 without movement and deformation. For this reason, the pseudo mask derivation unit 22 deforms the three-dimensional model 41 matching the shapes of the masks applied to the fixed submucosal layer and muscularis propria in the vicinity of the rectal cancer. With this, it is possible to derive the pseudo mask image Mf0 representing the rectal cancer having a natural shape matching the shape of the rectum.

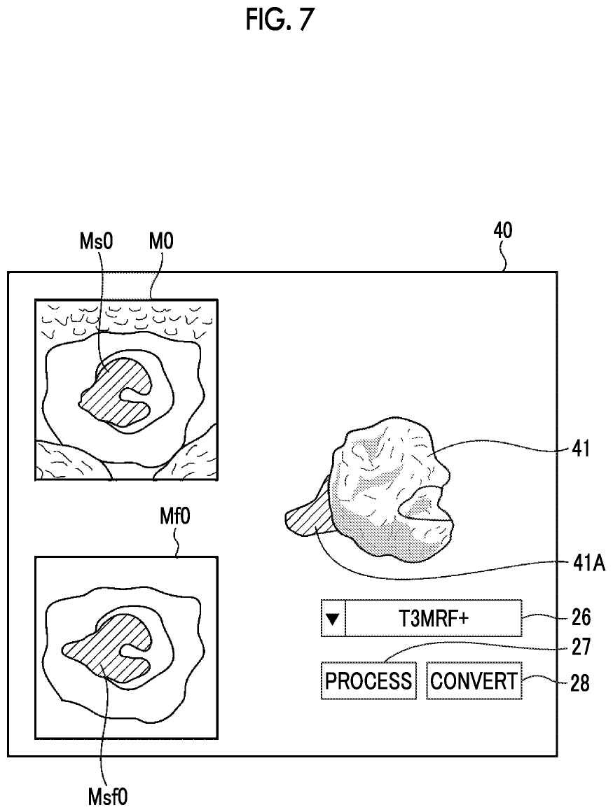

FIG. 7 shows a state in which, on the mask processing screen 40, the three-dimensional model 41 is deformed to represent the rectal cancer of the stage T3MRF+ from the rectal cancer of the stage T3ab and a region 41A is added. With the deformation of the three-dimensional model 41, the mask Msf0 obtained by processing the mask Ms0 of the mask image M0 is applied to the pseudo mask image Mf0.

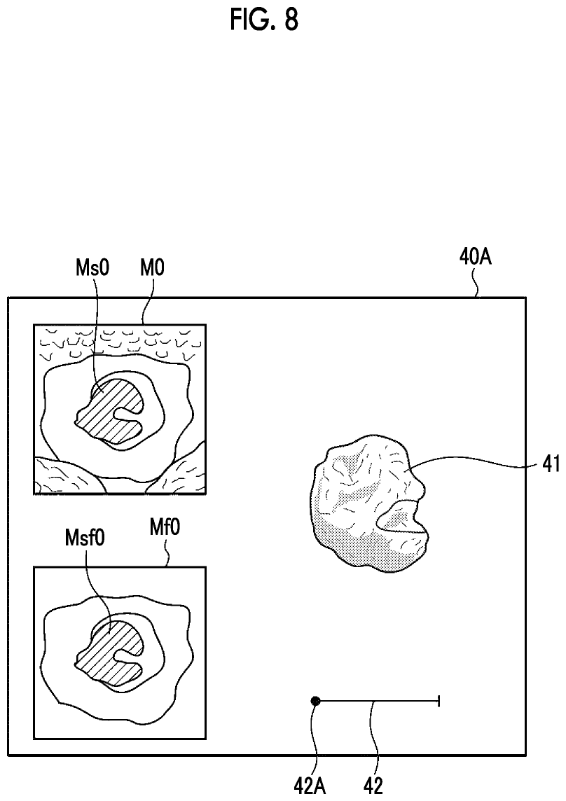

The operator may instruct the degree of deformation of the three-dimensional model 41. FIG. 8 is a diagram showing a mask processing screen on which the operator can instruct the degree of deformation of the three-dimensional model. In FIG. 8, the same components as those in FIG. 6 are represented by the same reference numerals, and here, detailed description will not be repeated. As shown in FIG. 8, on a mask processing screen 40A, a scale 42 for adjusting the degree of deformation of the three-dimensional model 41 is displayed, instead of the pull-down menu 26 and the PROCESS button 27 shown in FIG. 6.

On the mask processing screen 40A shown in FIG. 8, the operator designates the degree of deformation of the three-dimensional model 41 by moving a knob 42A of a scale 42 using the input device 15. With this, the pseudo mask derivation unit 22 deforms the three-dimensional model 41 to extend with a designated position as a starting point, and processes the mask Msf0 applied to the pseudo mask image Mf0 to conform to the shape of the extended three-dimensional model 41. Even in this case, the pseudo mask derivation unit 22 deforms the three-dimensional model 41 such that a shape and/or a progress of a lesion is different from that of a lesion included in the original image G0 based on a lesion shape evaluation index used as an evaluation index in medical practice for a medical image designated by the operator. The pseudo mask derivation unit 22 processes the mask while maintaining the three-dimensional continuity of the mask applied to the rectal cancer.

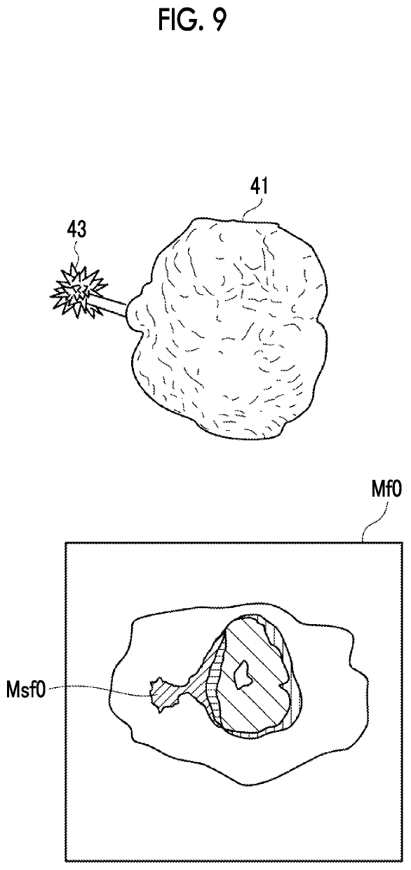

The derivation of the pseudo mask image Mf0 is not limited to that depending on the stage of the rectal cancer designated as described above. For example, in addition to or instead of the pull-down menu 26 for selecting the stage of the rectal cancer, a pull-down menu for selecting the presence or absence of application of a spinous protrusion may be displayed on the mask processing screen 40 shown in FIG. 6 to generate a mask having a plurality of spinous protrusions, such as an infiltrated lymph node. In addition to the scale 42 shown in FIG. 8, a pull-down menu for selecting the presence or absence of application of a spinous protrusion may be displayed. In this case, the three-dimensional model 41 of the mask processing screen 40 is a three-dimensional model in which an infiltrated lymph node 43 is applied as shown in FIG. 9, and the pseudo mask image Mf0 is a pseudo mask image in which a region of the infiltrated lymph node 43 is added to the mask Msf0 of the rectal cancer.

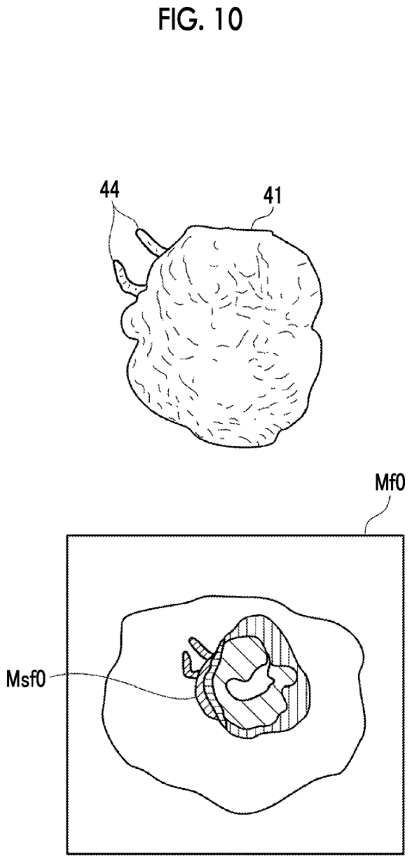

In addition to or instead of the pull-down menu 26 for selecting the stage of the rectal cancer, a pull-down menu for selecting the presence or absence of application of a protrusion may be displayed on the mask processing screen 40 shown in FIG. 6 to generate a mask having a small protrusion, such as vascular infiltration. In addition to the scale 42 shown in FIG. 8, a pull-down menu for selecting the presence or absence of application of a protrusion may be displayed. The operator can designate a position in the rectal cancer where a protrusion is applied and a distal end position of the protrusion using the input device 15. In this case, the three-dimensional model 41 of the mask processing screen 40 is a three-dimensional model in which the position where the protrusion is applied and the distal end position of the protrusion are interpolated by spline interpolation or the like as shown in FIG. 10, and vascular infiltrations 44 are applied. In the three-dimensional model 41 shown in FIG. 10, two vascular infiltrations are applied. The pseudo mask image Mf0 is a pseudo mask image in which regions of the two vascular infiltrations 44 are added to the mask of the rectal cancer.

The pseudo mask derivation unit 22 derives information representing the stage of the rectal cancer on the derived pseudo mask image Mf0. Here, since the stage of the rectal cancer is selected from the pull-down menu 26 by the operator on the mask processing screen 40 shown in FIG. 6, the selected stage of the rectal cancer may be used without change. On the other hand, on the mask processing screen 40A shown in FIG. 8, the degree of processing is instructed by the operator. For this reason, the pseudo mask derivation unit 22 determines the stage depending on a depth of the mask Msf0 from the mucous membrane layer 31 on the rectal cancer included in the pseudo mask image Mf0, and applies information representing the stage to the pseudo mask image Mf0. Information representing the stage of the rectal cancer in the pseudo mask image Mf0 may be information based on an input from the input device 15 by the operator.

In a case where the CONVERT button 28 is selected on the mask processing screen 40, the pseudo image derivation unit 23 derives a pseudo image having a region based on the mask included in the pseudo mask image Mf0, based on the original image G0 and the pseudo mask image Mf0. To this end, the pseudo image derivation unit 23 has a generator 50 that performs learning using generative adversarial networks (GAN). The generator 50 is constructed by performing learning to output a pseudo image having a region based on a mask included in the mask image in a case where the original image G0 including the rectal cancer and the mask image are input.

In the present embodiment, the pseudo image means an image having the same representation format as an image acquired by a modality that acquires the original image G0. That is, in a case where the original image G0 is an MRI image acquired by an MRI imaging apparatus, the pseudo image means an image having the same representation format as the MRI image. Here, the same representation format means that structures having the same composition are represented with the same density or brightness.

Here, as the generator 50, for example, a generator constructed by a technique of SPADE for generating a pseudo image from a mask image, described in “Semantic Image Synthesis with Spatially-Adaptive Normalization, Park, et al., arXiv:1903.07291v2 [cs.CV] 5 Nov. 2019” can be used.

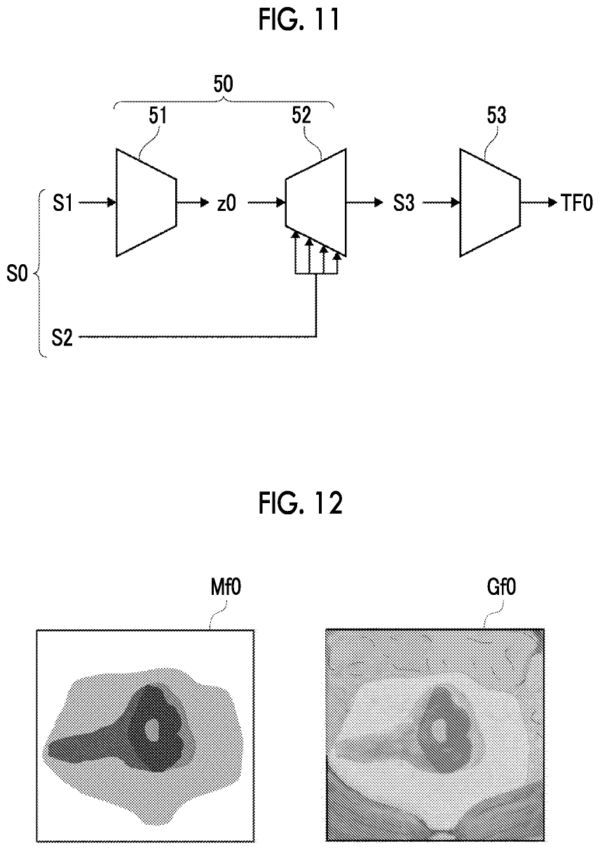

FIG. 11 is a diagram schematically showing a generator and learning thereof. As shown in FIG. 11, the generator 50 has an encoder 51 and a decoder 52. In the present embodiment, the generator 50 configures generative adversarial networks (GAN) along with a discriminator 53 described below. In the example shown in FIG. 11, training data S0 composed of an image 51 for learning that is an MRI image including a rectum and a mask image S2 for learning in which masks are applied to a rectal cancer, a mucous membrane layer, a submucosal layer, a muscularis propria, and a subserous layer in the image 51 for learning is prepared.

The encoder 51 that configures the generator 50 is composed of a convolutional neural network (CNN) that is one multilayered neural network in which a plurality of processing layers are connected hierarchically, and in the present embodiment, outputs a latent representation z0 representing a feature quantity of the MRI image including the rectum in a case where the image 51 for learning is input.

The decoder 52 applies a mask of an individual region included in the mask image S2 for learning to generate a region represented by each mask while decoding the latent representation z0 output from the encoder 51, and outputs a pseudo image S3 having a region based on each of the masks included in the mask image and has the same representation format as the image 51 for leaning.

The discriminator 53 discriminates whether the input image is a real image or the pseudo image generated by the generator 50, and outputs a discrimination result TF0. Here, the real image is not an image generated by the generator 50, but an original image acquired by imaging a subject by the imaging apparatus 3. In contrast, the pseudo image is an image having the same representation format as the original image, generated from the mask image by the generator 50.

In the present embodiment, learning of the discriminator 53 is performed to correctly discriminate the discrimination result TF0 regarding whether the input image is a real image or the pseudo image generated by the generator 50. Learning of the generator 50 is performed to derive a pseudo image similar to the real image from the input mask image and is performed such that the discriminator 53 incorrectly discriminates the discrimination result TF0. With this, the generator 50 can generate a pseudo image having the same representation format as an MRI image of a real thing, not identified by the discriminator 53.

The pseudo image derivation unit 23 derives a pseudo image from the original image G0 and the pseudo mask image Mf0 derived by the pseudo mask derivation unit 22 with the generator 50 constructed in this manner. For example, in a case where the original image G0 and the pseudo mask image Mf0 shown in FIG. 12 are input, the generator 50 outputs a pseudo image Gf0 having the same representation format as the original image G0. The derived pseudo image Gf0 is stored in the storage 13 in conjunction with the pseudo mask image Mf0 and information representing the stage of the rectal cancer. Here, in the present embodiment, the existing images that are not generated by the image generation apparatus according to the present embodiment, that is, the original image G0, the mask image M0 in which the mask is applied to the rectal cancer of the original image G0, and information representing the stage of the rectal cancer are accumulated as training data for learning a segmentation model described below in the storage 13. Then, in the present embodiment, the pseudo image Gf0, the pseudo mask image Mf0, and information representing the stage of the rectal cancer that are derived by the image generation apparatus 1 according to the present embodiment are accumulated as training data in the storage 13, in addition to existing training data. The pseudo image Gf0, the pseudo mask image Mf0, and information representing the stage of the rectal cancer may be transmitted to the image storage server 4 and may be accumulated in conjunction with the existing training data.

The learning unit 24 learns the segmentation model that segments the MM image including the rectum into a plurality of regions. In the present embodiment, learning of a semantic segmentation model that segments the MM image into the regions of the rectal cancer, the mucous membrane layer of the rectum, the submucosal layer of the rectum, the muscularis propria of the rectum, the subserous layer of the rectum, and the background other than the layers is performed, and a trained semantic segmentation model is constructed. The semantic segmentation model (hereinafter, referred to as an SS model) is a machine learning model that outputs an output image with a mask representing an extraction target object (class) applied to each pixel of an input image as well known in the art. In the present embodiment, the input image to the SS model is the MRI image including the region of the rectum, and the output image is the mask image in which the regions of the rectal cancer, the mucous membrane layer of the rectum, the submucosal layer of the rectum, the muscularis propria of the rectum, the subserous layer of the rectum, and the background other than the layers in the MRI image are masked. The SS model is constructed by a convolutional neural network (CNN), such as Residual Networks (ResNet) or U-shaped Networks (U-Net).

In regard to the learning of the SS model, in addition to existing training data, that is, training data composed of a combination of the original image G0 and the mask image M0 of the original image G0, training data composed of a combination of the pseudo mask image Mf0 derived by the pseudo mask derivation unit 22 and the pseudo image Gf0 derived by the pseudo image derivation unit 23 is used. In the existing training data, the original image G0 and the pseudo image Gf0 are data for learning, and the mask image M0 and the pseudo mask image Mf0 are correct answer data. In the training data including the pseudo image Gf0 and the pseudo mask image Mf0, the pseudo image Gf0 is data for learning, and the pseudo mask image Mf0 is correct answer data.

The original image G0 and the pseudo image Gf0 are input to the SS model at learning, and a mask image in which an object included in the images is segmented is output. Next, a difference between the mask image output from the SS model, and the mask image M0 and the pseudo mask image Mf0 as correct answer data is derived as a loss. Then, learning of the SS model is repeated using a plurality of kinds of training data such that the loss decreases, and the SS model is constructed.

The learning unit 24 performs learning of a discrimination model that discriminates the stage of the rectal cancer on the MRI image including the rectum, and a trained discrimination model is constructed. In the present embodiment, an input image to the discrimination model is the MRI image including the regions of the rectum and a mask image obtained by segmenting the MRI image, and an output is the stage of the rectal cancer included in the MM image. The discrimination model is also constructed by a convolutional neural network, such as ResNet or U-Net.

At learning of the discrimination model, in addition to existing training data, that is, training data composed of a combination of the existing training data, that is, the original image G0, the mask image M0 of the original image G0, and information representing the stage of the rectal cancer included in the original image G0, training data composed of a combination of the pseudo mask image Mf0 derived by the pseudo mask derivation unit 22, the pseudo image Gf0 derived by the pseudo image derivation unit 23, and the information representing the stage of the rectal cancer included in the pseudo image Gf0 is used. In the existing training data, the original image G0 and the mask image M0 are data for learning, and information representing the stage of the rectal cancer of the original image G0 is correct answer data. In the training data including the pseudo image Gf0 and the pseudo mask image Mf0, the pseudo image Gf0 and the pseudo mask image Mf0 are data for learning, and information representing the stage of the rectal cancer is correct answer data.

The original image G0 and the mask image M0, and the pseudo image Gf0 and the pseudo mask image Mf0 are input to the discrimination model at learning, and information representing the stage of the rectal cancer included in such images is output. As information representing the stage of the rectal cancer, a probability of each stage of the rectal cancer is used. The probability has a value of 0 to 1. Next, a difference between the probability of each stage of the rectal cancer and the stage of the rectal cancer of correct answer data is derived as a loss. Here, in a case where it is assumed that the stage of the rectal cancer output from the discrimination model is (T1, T2, T3, T4)=(0.1, 0.1, 0.7, 0.1), and correct answer data is (0, 0, 1, 0), the difference between the probability of each stage of the rectal cancer output from the discrimination model and the probability of each stage of the rectal cancer in correct answer data is derived as a loss. Then, learning of the discrimination model is repeated using a plurality of kinds of training data such that the loss decreases, and the discrimination model is constructed.

The discrimination model may be constructed to output information representing the stage of the rectal cancer included in the input image in a case where only the mask image on the input image is input. In this case, in learning of the discrimination model, training data including the mask image M0 on the original image G0 as data for learning and information representing the stage of the rectal cancer included in the original image G0 as correct answer data, and training data including the pseudo mask image Mf0 on the pseudo image Gf0 as data for learning and information representing the stage of the rectal cancer included in the pseudo image Gf0 as correct answer data are used.

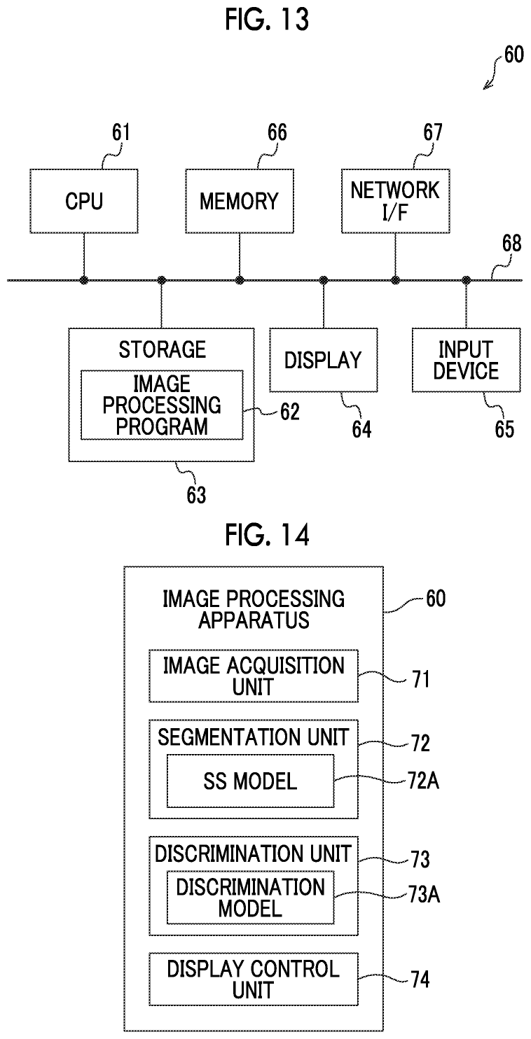

Next, the image processing apparatus according to the present embodiment will be described. FIG. 13 is a diagram showing the hardware configuration of the image processing apparatus according to the present embodiment. As shown in FIG. 13, an image processing apparatus 60 includes a CPU 61, a non-volatile storage 63, and a memory 66 as a temporary storage region. The image processing apparatus 60 includes a display 64, such as a liquid crystal display, an input device 65, such as a keyboard and a mouse, and a network interface (I/F) 67 that is connected to the network 5. The CPU 61, the storage 63, the display 64, the input device 65, the memory 66, and the network I/F 67 are connected to a bus 68. The CPU 61 is an example of a processor in the present disclosure.

An image processing program 62 is stored in the storage 63. The CPU 61 reads out the image processing program 62 from the storage 63, develops the image processing program 62 to the memory 66, and executes the developed image processing program 62.

Next, the functional configuration of the image processing apparatus according to the present embodiment will be described. FIG. 14 is a diagram showing the functional configuration of the image processing apparatus according to the present embodiment. As shown in FIG. 14, the image processing apparatus 60 comprises an image acquisition unit 71, a segmentation unit 72, a discrimination unit 73, and a display control unit 74. Then, the CPU 61 executes the image processing program 62, whereby the CPU 61 functions as the image acquisition unit 71, the segmentation unit 72, the discrimination unit 73, and the display control unit 74.

The image acquisition unit 71 acquires a target image T0 to be a target of processing from the image storage server 4. The target image T0 is an MRI image including a rectum of a patient.

The segmentation unit 72 segments regions of an object included in the target image T0 to derive a mask image TM0 in which the regions of the object included in the target image T0 are masked. In the present embodiment, the mask image TM0 in which regions of a rectal cancer, a mucous membrane layer of a rectum, a submucosal layer of the rectum, a muscularis propria of the rectum, a subserous layer of the rectum, and a background other than the layers included in the target image T0 are segmented, and a mask is applied to each region is derived. To this end, an SS model 72A constructed by the learning apparatus according to the present embodiment is applied to the segmentation unit 72.

The discrimination unit 73 discriminates a stage of the rectal cancer included in the target image T0 and outputs a discrimination result. To this end, a discrimination model 73A constructed by the learning apparatus according to the present embodiment is applied to the discrimination unit 73. The target image T0 and the mask image TM0 of the target image T0 derived by the segmentation unit 72 are input to the discrimination model 73A, and the discrimination result of the stage of the rectal cancer included in the target image T0 is output.

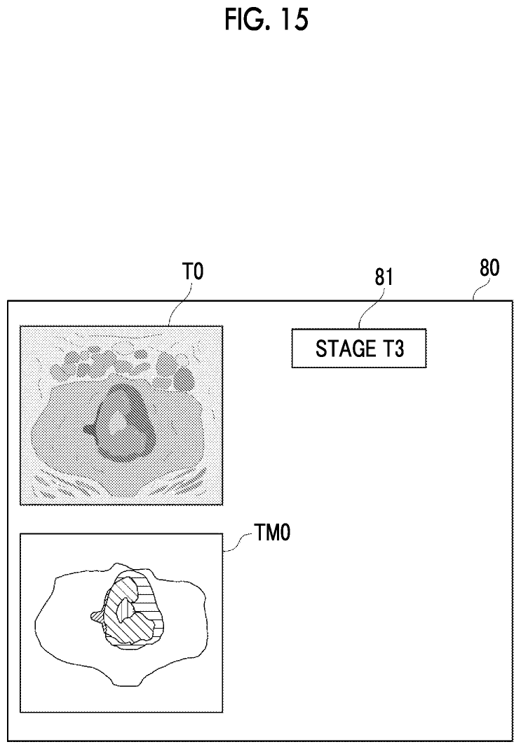

The display control unit 74 displays the mask image TM0 derived by the segmentation unit 72 and the discrimination result of the stage of the rectal cancer derived by the discrimination unit 73 on the display 64. FIG. 15 is a diagram showing a display screen of the mask image TM0 and the discrimination result. As shown in FIG. 15, the target image T0, the mask image TM0, and a discrimination result 81 are displayed on a display screen 80. In FIG. 15, the discrimination result is “STAGE T3”.

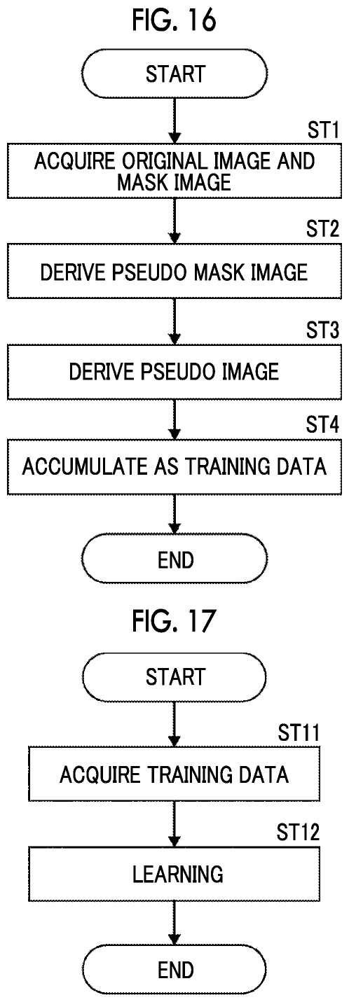

Next, processing that is executed in the present embodiment will be described. FIG. 16 is a flowchart of image generation processing in the present embodiment. First, the information acquisition unit 21 acquires the original image G0 and the mask image M0 from the image storage server 4 (Step ST1). Next, the pseudo mask derivation unit 22 derives the pseudo mask image Mf0 by processing the mask (Step ST2). Then, the pseudo image derivation unit 23 derives the pseudo image Gf0 having the region based on the pseudo mask (Step ST3), accumulates the pseudo image Gf0, the pseudo mask image Mf0, and information representing the stage of the rectal cancer included in the pseudo image Gf0 as training data in the storage 13 or the image storage server 4 in conjunction with existing training data (Step ST4), and the processing ends.

FIG. 17 is a flowchart of learning processing in the present embodiment. First, the learning unit 24 acquires the training data composed of a combination of the pseudo image Gf0 and the pseudo mask image Mf0 (Step ST11). Then, the learning unit 24 performs learning of the SS model using the training data (Step ST12). With this, the trained SS model is constructed.

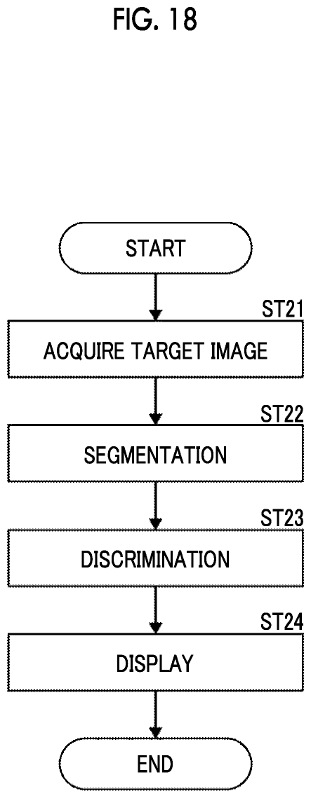

FIG. 18 is a flowchart of image processing in the present embodiment. First, the image acquisition unit 71 acquires the target image T0 to be a target of processing from the image storage server 4 (Step ST21). Then, the segmentation unit 72 segments the target image T0 by the SS model 72A to derive the mask image TM0 (Step ST22). Next, the discrimination unit 73 derives the discrimination result of the stage of the rectal cancer included in the target image T0 by the discrimination model 73A (Step ST23). Then, the display control unit 74 displays the display screen of the mask image TM0 and the discrimination result (Step ST24), and the processing ends.

Here, since a scarce disease having a small frequency of appearance, such as a progressive cancer, has a small number of cases, it is not possible to prepare a sufficient amount of training data for constructing a machine learning model for performing segmentation and stage discrimination. For this reason, it is difficult to provide a machine learning model capable of accurately segmenting a scarce disease or accurately discriminating a class of a scarce disease, such as a stage of a progressive cancer.

In the present embodiment, the pseudo mask image Mf0 is derived by processing the mask in the mask image M0 on the original image G0, and the pseudo image Gf0 having the region based on the pseudo mask image Mf0 is derived. With this, it is possible to prepare training data including a target object of a class that is not in existing training data for constructing a segmentation model or is not at all. For example, a pseudo image Gf0 including a progressed rectal cancer can be prepared as training data. For this reason, a pseudo image Gf0 on a scarce disease is derived, is accumulated along with existing training data, and is used for learning of a learning model for performing segmentation and stage discrimination, whereby a sufficient amount of training data to such an extent that segmentation can be accurately performed even on a scarce disease can be prepared. Accordingly, it is possible to provide a machine learning model capable of accurately segmenting a scarce disease or accurately discriminating a class of a scarce disease, such as a stage of a progressive cancer, on a target image to be processed.

In the above-described embodiment, although the target object is the rectal cancer, the present disclosure is not limited thereto. A lesion, such as a cancer or a tumor of an organ or a structure other than the rectum can be used as a target object. For example, derivation of a pseudo mask and a pseudo image and construction of an SS model with a bone spur in a joint as a target object can be performed. Hereinafter, this will be described as another embodiment.

Here, the bone spur refers to a disease that a cartilage of an articular facet hypertropically grows and is gradually hardened and ossified, and becomes like a “spur”, and is one of characteristic findings of osteoarthritis to be expected around an articular facet. In such a case, a pseudo mask may be derived to form a bone spur with a bone composing a joint as a target object, and a pseudo image in which the bone spur is formed on the bone composing the joint may be derived.

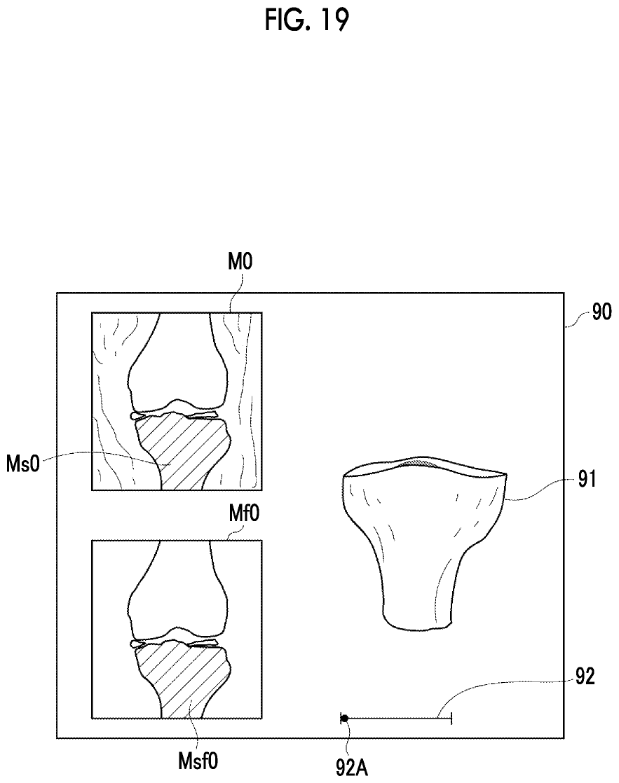

FIG. 19 is a diagram showing a mask processing screen on the bone spur. As shown in FIG. 19, on a mask processing screen 90, a mask image M0, a pseudo mask image Mf0 in which a mask in the mask image M0 is processed, a three-dimensional model 91 of a knee joint included in an original image G0, and a scale 92 for adjusting a degree of deformation of the three-dimensional model 91 are displayed. Here, the original image G0 is an MM image of a knee joint of a patient. The three-dimensional model 91 of the knee joint represents a joint of a shinbone. In the original image G0, a bone spur is not formed on the joint.

Here, for description, a mask Ms0 is applied to only a region of the shinbone to be processed in the mask image M0 shown in FIG. 19. In the pseudo mask image Mf0, only the mask Msf0 of the shinbone to be processed is represented by the reference numeral. Since the mask image M0 and the pseudo mask image Mf0 shown in FIG. 19 are before mask processing, the masks applied to the respective images are the same.

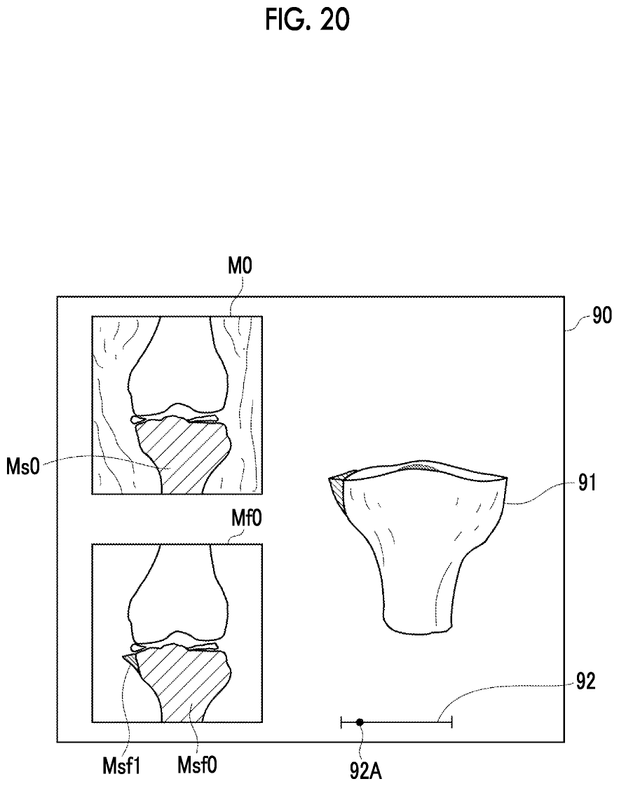

The three-dimensional model 91 is a three-dimensional image derived by extracting only a region near the joint of the shinbone in the original image G0 and performing volume rendering. The operator can change omnidirectional orientations of the three-dimensional model 91 by operating the input device 15. The operator designates a processing place of the mask by designating a desired position in the three-dimensional model 91 using the input device 15. Then, the operator designates a degree of processing of the mask by moving a knob 92A of the scale 92 using the input device 15. With this, the pseudo mask derivation unit 22 processes the three-dimensional model 91 to extend with the designated position as a starting point as shown in FIG. 20, and applies a mask Msf1 to conform to the shape of the three-dimensional model 91 with respect to the mask Msf0 applied to the pseudo mask image Mf0.

In this case, the pseudo mask derivation unit 22 processes the mask while maintaining three-dimensional continuity of the mask applied to the shinbone. For example, in deforming to extend the designated position in the three-dimensional model 91, the degree of deformation decreases as far from the designated position in the three-dimensional model 91. With this, it is possible to deform the three-dimensional model 91 while maintaining three-dimensional continuity in the original three-dimensional model 91, and as a result, it is possible to process the mask like the shinbone on which the bone spur is formed while maintaining the three-dimensional continuity of the mask applied to the shinbone.

The pseudo image derivation unit 23 derives the pseudo image Gf0 including the shinbone on which the bone spur is formed, from the pseudo mask image Mf0 in which the bone spur is formed. Then, the learning unit 24 learns the SS model using the pseudo mask image WO in which the bone spur is formed and the pseudo image Gf0 as training data. With this, it is possible to construct a trained SS model capable of accurately segmenting the bone spur in the MRI image including the knee joint. Accordingly, the SS model constructed in this manner is applied to the segmentation unit 72 of the image processing apparatus according to the present embodiment, whereby it is possible to accurately segment the region of the bone spur in the MRI image including the knee joint.

The pseudo image Gf0 including the shinbone on which the bone spur is formed in the joint is used as training data, whereby it is possible to construct a discrimination model that discriminates the presence or absence of the bone spur, on the MRI image including the joint of the shinbone. In constructing the discrimination model that discriminates the presence or absence of the bone spur, the original image including the shinbone on which no bone spur is formed is also used as training data.

In another embodiment described above, although the pseudo mask image Mf0 and the pseudo image Gf0 are derived by processing the original image G0 in which no bone spur is included, a lesion to be added is not limited to the bone spur. An organ to be a target of addition of any lesion may be included, and an original image G0 may be processed to add the lesion to the original image G0 including no lesion, thereby deriving a pseudo mask image Mf0 and a pseudo image Gf0.

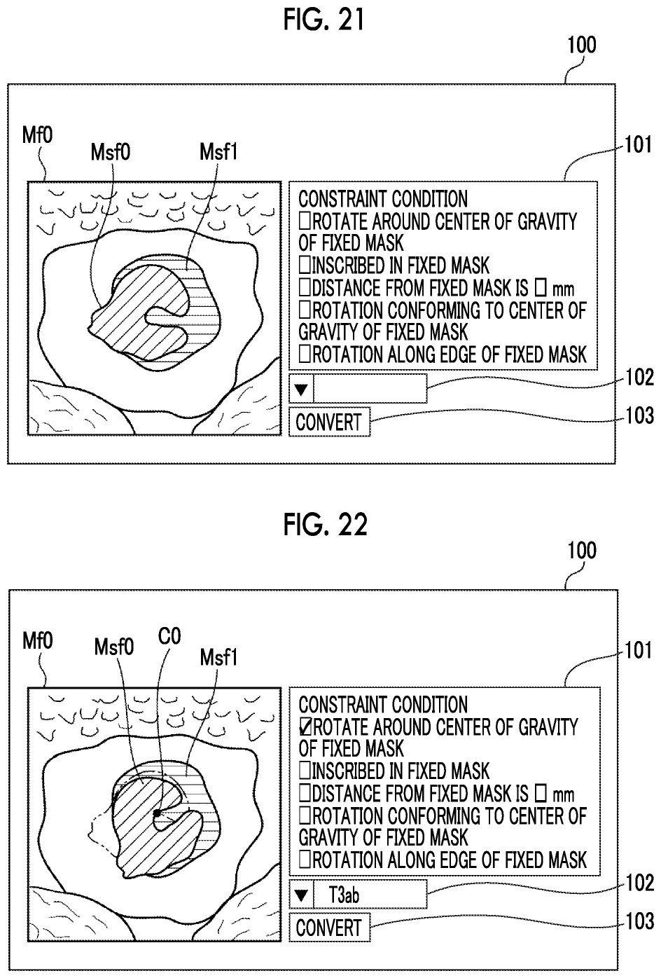

In each embodiment described above, in a case where the pseudo mask derivation unit 22 processes the mask to derive the pseudo mask image WO, constraint conditions may be set with respect to the deformation of the mask, and designation of the degree of processing of the mask may be received under the constraint conditions. FIG. 21 is a diagram showing another example of a mask processing screen. On a mask processing screen 100 shown in FIG. 21, a pseudo mask image WO, a condition list 101 for setting constraint conditions, a pull-down menu 102 for setting the degree of deformation of a mask, and a CONVERT button 103 for executing a derivation of a pseudo image are displayed.

In the pseudo mask image Mf0 displayed on the mask processing screen 100 shown in FIG. 21, a mask Msf0 is applied to a region of a rectal cancer, and a mask Msf1 is applied to a region of a rectum. In regard to the masks Msf0 and Msf1 that are included in the pseudo mask image Mf0 displayed on the mask processing screen 100, it is possible to set whether or not the process the mask. For example, through a predetermined operation to move a mouse cursor on a desired mask and right-click, or the like, it is possible to set whether or not the process the selected mask. In the present embodiment, it is assumed that the mask Msf1 of the rectum is not processed, and the mask Msf0 of the rectal cancer is set to process. In the following description, the mask that is set not to process is referred to as a fixed mask.

In the condition list 101, constraint conditions regarding deformation of a mask to be processed are displayed to be selectable by checkboxes. As shown in FIG. 21, as the constraint conditions, for example, “ROTATE AROUND CENTER OF GRAVITY OF FIXED MASK”, “INSCRIBED IN FIXED MASK”, “DISTANCE FROM FIXED MASK IS □ mm”, “ROTATION CONFORMING TO CENTER OF GRAVITY OF FIXED MASK”, and “ROTATION ALONG EDGE OF FIXED MASK” are displayed. The operator can select one or more desired constraint conditions from the condition list 101. In regard to the constraint condition “DISTANCE FROM FIXED MASK IS □ mm”, a numerical value of a distance can be input. In the present embodiment, it is assumed that “ROTATE AROUND CENTER OF GRAVITY OF FIXED MASK” is selected as the constraint condition. Like the pull-down menu 26 shown in FIG. 6, in the pull-down menu 102, the stages T1, T2, T3ab, T3cd, T3MRF+, T4a, and T4b of the rectal cancer can be selected.

In a case where “ROTATE AROUND CENTER OF GRAVITY OF FIXED MASK” is selected as the constraint condition, a center C0 of gravity of the mask Msf1 of the rectum as the fixed mask is displayed in the pseudo mask image Mf0. With this, in a case where the operator deforms and processes the mask Mfs0 using the input device 15, the deformation of the mask Msf0 is constrained such that only rotation around the center C0 of gravity of the fixed mask Msf1 is possible. FIG. 22 shows a state in which the mask Msf0 is rotated. In FIG. 22, the mask Msf0 before deformation is indicated by a virtual line. The operator can select the stage of the rectal cancer in the pull-down menu 102. In FIG. 22, T3ab that is the same stage as the rectal cancer currently displayed is displayed.

Then, after the mask Msf0 is processed and the pseudo mask image Mf0 is derived, in a case where the CONVERT button 103 is selected, the pseudo image derivation unit 23 derives the pseudo image Gf0 depending on the processed mask Msf0 and the selected stage of the rectal cancer.

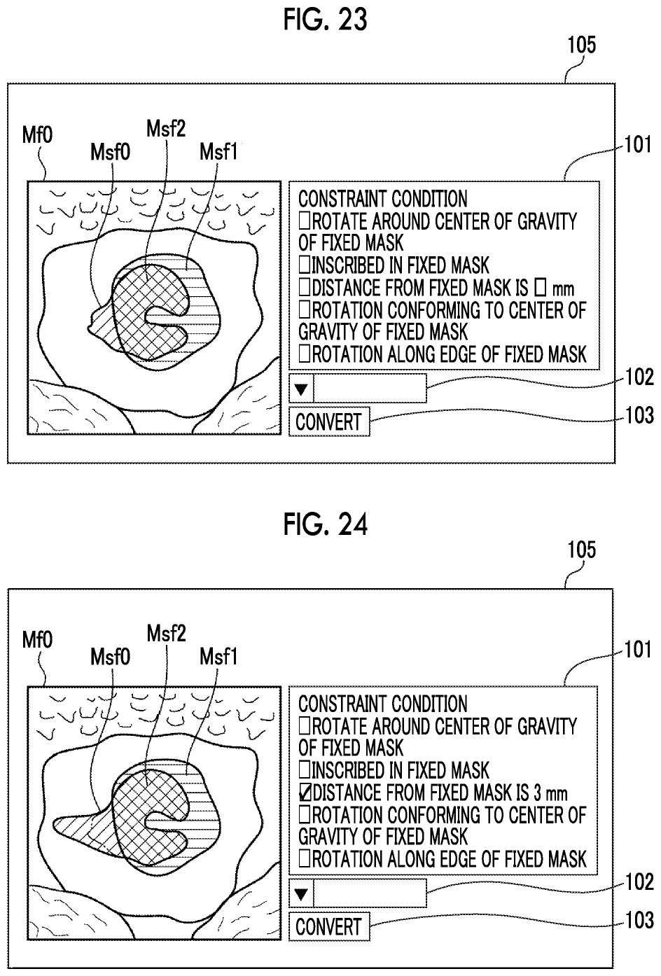

FIG. 23 is a diagram showing another example of a mask processing screen. In a pseudo mask image Mf0 displayed on a mask processing screen 105 shown in FIG. 23, a region in the mask Mfs0 shown in FIG. 21 overlapping the rectum is set as a fixed mask Mfs2. With this, only the mask Mfs0 that is present in a region outside the rectum can be deformed. Here, it is assumed that “DISTANCE FROM FIXED MASK IS □ mm” is selected as the constraint condition, and 3 mm is input as the distance. With this, in a case where the operator deforms and processes the mask Mfs0 using the input device 15, the deformation of the mask Msf0 is constrained such that a position farthest from the fixed mask is equal to or less than 3 mm. FIG. 24 shows a state in which the mask Msf0 is deformed. As shown in FIG. 24, a protruding portion in the mask Msf0 is deformed such that the distance from each of the fixed mask Msf1 and Msf2 is the designated distance (that is, 3 mm). The contour of the mask Msf0 before deformation is indicated by a virtual line.

Then, after the mask Msf0 is processed and the pseudo mask image Mf0 is derived, in a case where the CONVERT button 103 is selected, the pseudo image derivation unit 23 derives the pseudo image Gf0 depending on the processed mask Msf0 and the selected stage of the rectal cancer.

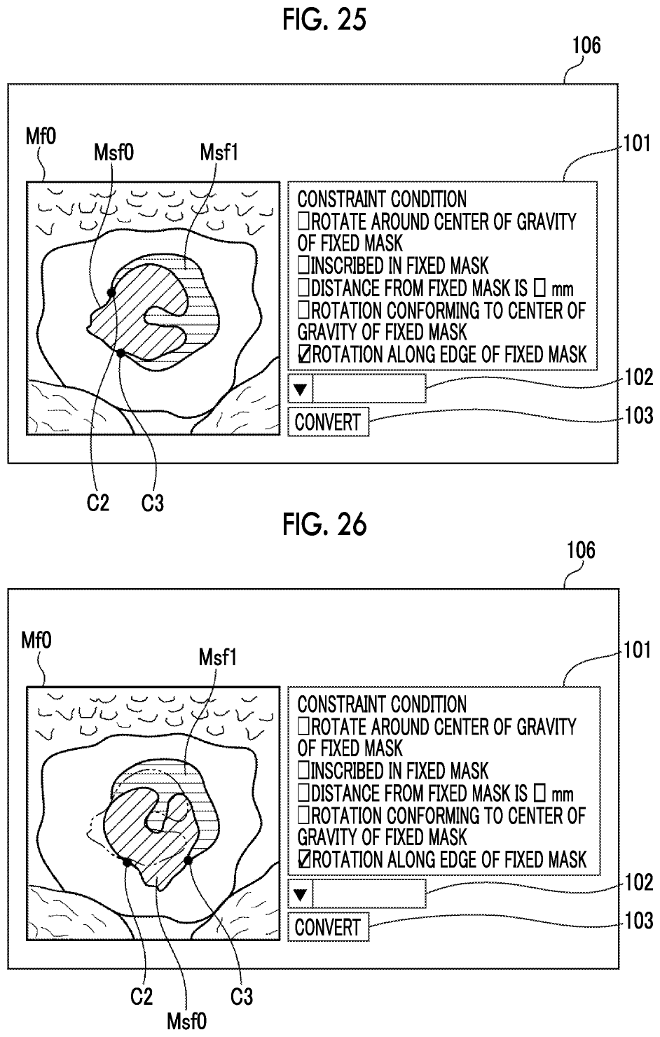

FIG. 25 is a diagram showing another example of a mask processing screen. In a mask image Mf0 displayed on a mask processing screen 106 shown in FIG. 25, the same masks Mfs0 and Msf1 as those in FIG. 21 are set. Then, it is assumed that, in the condition list 101, “ROTATION ALONG EDGE OF FIXED MASK” is selected. In a case where “ROTATION ALONG EDGE OF FIXED MASK” is selected in the In condition list 101, intersections C2 and C3 between the contour of the mask Msf1 of the rectum as the fixed mask and the contour of the mask Msf0 are displayed in the pseudo mask image Mf0. With this, in a case where the operator deforms and processes the mask Mfs0 using the input device 15, the deformation of the mask Msf0 is constrained such that only rotation along an edge of the fixed mask Msf1 is possible. FIG. 26 shows a state in which the mask Msf0 is rotated.

Then, after the mask Msf0 is processed and the pseudo mask image Mf0 is derived, in a case where the CONVERT button 103 is selected, the pseudo image derivation unit 23 derives the pseudo image Gf0 depending on the processed mask Msf0 and the selected stage of the rectal cancer.

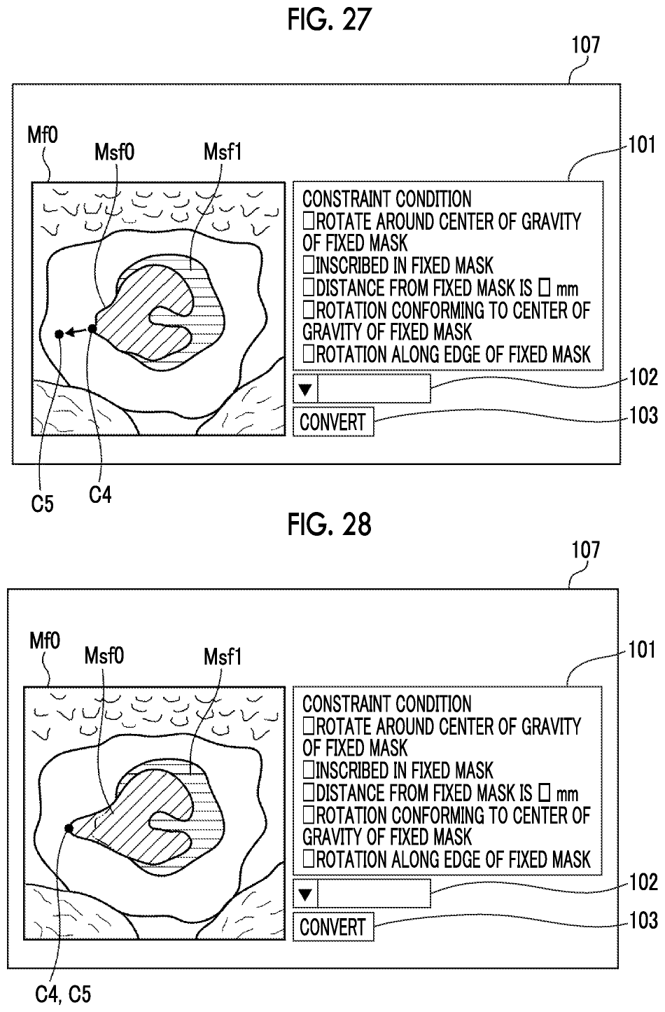

FIG. 27 is a diagram showing another example of a mask processing screen. Although the mask Msf0 is deformed using the scale 42 on the mask processing screen shown in FIG. 8 described above, on a mask processing screen 107 shown in FIG. 27, the operator operates the input device 15 to directly deform the mask Msf0. In this case, as shown in FIG. 28, the operator can deform and process the mask Msf0 by dragging a point C4 of an end portion of the mask Msf0 toward a point C5 using the input device 15. In this case, instead of dragging the point C4, only the point C5 may be designated using the input device 15. For example, a position of the point C5 may be designated by double-clicking the position of the point C5. In this case, the mask Msf0 is deformed and processed as shown in FIG. 28 along with the designation of the point C5.

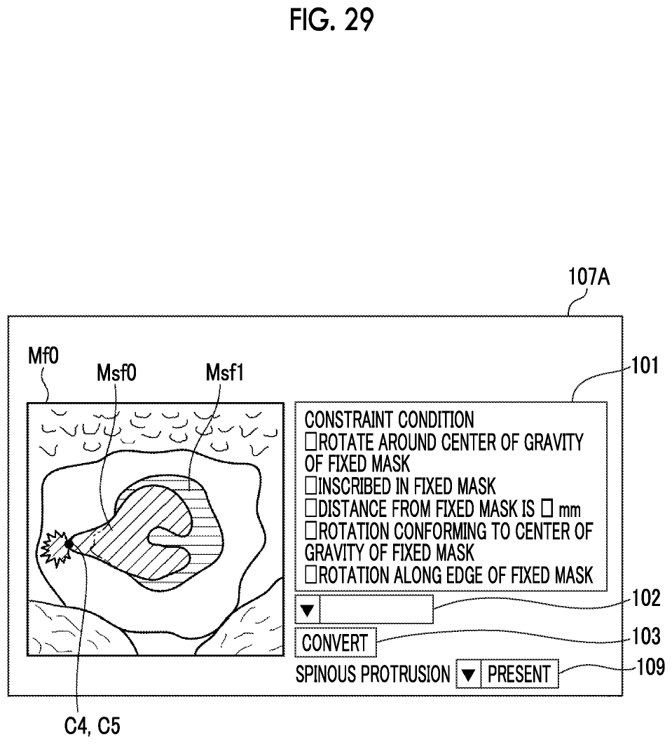

On the mask processing screen 107 shown in FIG. 28, a shape of a protruding end of the deformed mask Msf0 may be set. For example, a pull-down menu for selecting the presence or absence of a spinous protrusion such that a mask having the spinous protrusion shown in FIG. 9 can be generated may be displayed. FIG. 29 is a diagram showing a mask processing screen on which a pull-down menu for selecting the presence or absence of a spinous protrusion is displayed. As shown in FIG. 29, in a case where “PRESENT” is selected in a pull-down menu 109 of a spinous protrusion, and in a case where the operator drags the point C4 of the end portion of the mask Msf0 toward the point C5, the mask Msf0 is deformed and processed such that a mask having a spinous protrusion at a distal end of the points C4 and C5 as a movement destination is added.

In each embodiment described above, although the pseudo mask image Mf0 is derived by deforming the rectal cancer to be increased or adding the bone spur, the present disclosure is not limited thereto. The technique of the present disclosure can also be applied to a case of deriving a pseudo image Gf0 on a disease like constriction.

For example, in a case of deriving a pseudo image Gf0 including a disease of vascular constriction, a pseudo mask image Mf0 is derived by processing a mask of a blood vessel of an original image G0 including no vascular constriction to be thinned, whereby it is possible to derive the pseudo image Gf0 including vascular constriction.

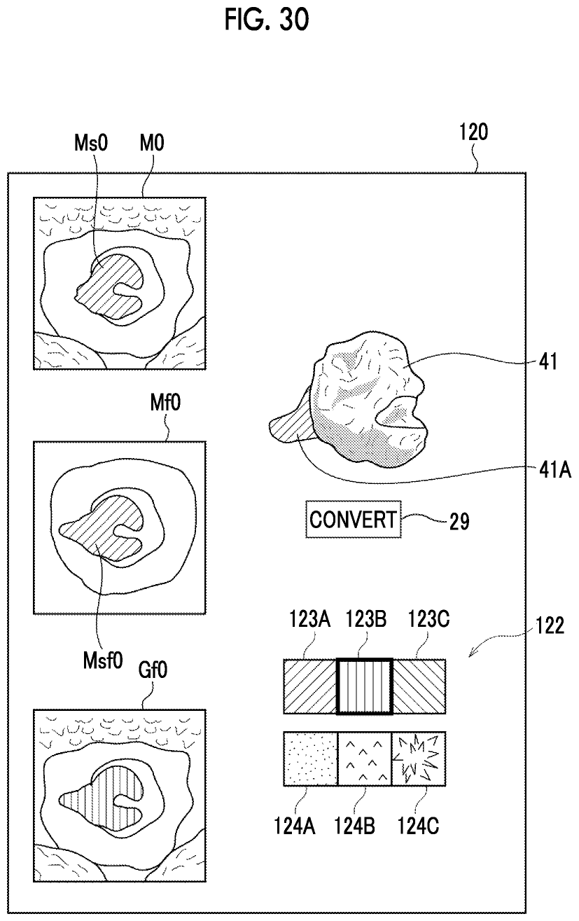

In the above-described embodiment, although the pseudo image derivation unit 23 derives the pseudo image Gf0 having the same representation format as the original image G0, the present disclosure is not limited thereto. In a case where the pseudo image derivation unit 23 derives a pseudo image, at least one of density, color, or texture of the pseudo image Gf0 may be changed. In this case, a plurality of style images having predetermined density, colors, and texture may be stored in the storage 13, and the pseudo image Gf0 may be derived to have the density, color, or texture selected from among a plurality of style images. FIG. 30 is a diagram showing a mask processing screen on which a plurality of style images are displayed. A mask processing screen 120 shown in FIG. 30 is composed by displaying a style image list 122 including a plurality of style images on the mask processing screen 40 shown in FIGS. 6 and 7 and displaying the pseudo image Gf0.

The style image list 122 displayed on the mask processing screen 120 shown in FIG. 30 includes style images of six different styles. An upper side of the list 122 displays style images 123A, 123B, 123C of three kinds of different colors, and a lower side displays style images 123A, 124B, 124C of three kinds of different texture. A style image of density may be included in addition to the color and texture or style images of two or all of the density, the color, and texture may be included. After the mask Msf0 is processed and the pseudo mask image Mf0 is derived, the operator selects style images of desired color and texture from the style image list 122 before selecting an CONVERT button 29. Here, it is assumed that the style image 123B of the color is selected. After the selection of the style image, in a case where the operator selects the CONVERT button 29, the pseudo image derivation unit 23 derives the pseudo image Gf0 including the rectal cancer of the color depending on the selected style image.

In the above-described embodiment, for example, as a hardware structure of processing units that execute various kinds of processing, such as the information acquisition unit 21, the pseudo mask derivation unit 22, the pseudo image derivation unit 23, and the learning unit 24 of the image generation apparatus 20, and the image acquisition unit 71, the segmentation unit 72, the discrimination unit 73, and the display control unit 74 of the image processing apparatus 60, various processors described below can be used. As described above, various processors include a programmable logic device (PLD) that is a processor capable of changing a circuit configuration after manufacturing, such as a field programmable gate array (FPGA), a dedicated electric circuit that is a processor having a circuit configuration dedicatedly designed for executing specific processing, such as an application specific integrated circuit (ASIC), and the like, in addition to a CPU that is a general-purpose processor configured to execute software (program) to function as various processing units.

One processing unit may be configured with one of various processors described above or may be configured with a combination of two or more processors (for example, a combination of a plurality of FPGAs or a combination of a CPU and an FPGA) of the same type or different types. A plurality of processing units may be configured with one processor.

As an example where a plurality of processing units are configured with one processor, first, as represented by a computer, such as a client or a server, there is a form in which one processor is configured with a combination of one or more CPUs and software, and the processor functions as a plurality of processing units. Second, as represented by system on chip (SoC) or the like, there is a form in which a processor that realizes all functions of a system including a plurality of processing units into one integrated circuit (IC) chip is used. In this way, various processing units are configured using one or more processors among various processors described above as a hardware structure.

In addition, as the hardware structure of various processors, more specifically, an electric circuit (circuitry), in which circuit elements, such as semiconductor elements, are combined can be used.

What is claimed is:

1. An image generation apparatus comprising:
at least one processor,
wherein the processor is configured to:
acquire an original image and a mask image in which masks are applied to one or more regions respectively representing one or more objects including a target object in the original image;
derive a pseudo mask image by processing the mask in the mask image; and
derive a pseudo image that has a region based on a mask included in the pseudo mask image and has the same representation format as the original image, based on the original image and the pseudo mask image.

2. The image generation apparatus according to claim 1,
wherein the pseudo mask image and the pseudo image are used as training data for learning a segmentation model that segments the object included in an image.

3. The image generation apparatus according to claim 2,
wherein the processor is configured to accumulate the pseudo mask image and the pseudo image as the training data.

4. The image generation apparatus according to claim 1,
wherein the processor is configured to derive the pseudo mask image that is able to generate the pseudo image including a target object of a class different from a class indicated by the target object.

5. The image generation apparatus according to claim 1,
wherein the processor is configured to derive the pseudo mask image by processing the mask such that at least one of a shape or a progress of a lesion is different from that of a lesion included in the original image, based on a lesion shape evaluation index used as an evaluation index in medical practice for a medical image.

6. The image generation apparatus according to claim 1,
wherein the processor is configured to derive the pseudo mask image by processing the mask until a normal organ has a shape to be evaluated as a lesion based on a measurement index in medical practice for a medical image.

7. The image generation apparatus according to claim 1,
wherein the processor is configured to refer to at least one style image having predetermined density, color, or texture and generate the pseudo image having density, color, or texture depending on the style image.

8. The image generation apparatus according to claim 1,
wherein the processor is configured to receive an instruction for a degree of processing of the mask and derive the pseudo mask image by processing the mask based on the instruction.

9. The image generation apparatus according to claim 8,
wherein the processor is configured to receive designation of a position of an end point of the mask after processing and designation of a processing amount as the instruction for the degree of processing.

10. The image generation apparatus according to claim 8,
wherein the processor is configured to receive the instruction for the degree of processing of the mask under a constraint condition set in advance.

11. The image generation apparatus according to claim 1,
wherein, in a case where the original image includes a plurality of the objects, and the target object and a partial region of another object other than the target object have an inclusion relation, in the mask image, a region having the inclusion relation is given with a mask different from a region having no inclusion relation.

12. The image generation apparatus according to claim 11,
wherein the processor is configured to, in a case where the other object having the inclusion relation is an object fixed in the original image, derive the pseudo mask image by processing the mask applied to the target object conforming to a shape of a mask applied to the fixed object.

13. The image generation apparatus according to claim 1,
wherein the processor is configured to, in a case where the original image is a three-dimensional image, derive the pseudo mask image by processing the mask while maintaining three-dimensional continuity of the mask applied to the region of the target object.

14. The image generation apparatus according to claim 1,
wherein the original image is a three-dimensional medical image, and
the target object is a lesion included in the medical image.

15. The image generation apparatus according to claim 14,
wherein the medical image includes a rectum of a human body, and
the target object is a rectal cancer, and another object other than the target object is at least one of a mucous membrane layer of the rectum, a submucosal layer of the rectum, a muscularis propria of the rectum, a subserous layer of the rectum, or a background other than the layers.

16. The image generation apparatus according to claim 14,
wherein the medical image includes a joint of a human body, and
the target object is a bone composing the joint, and another object other than the target object is a background other than the bone composing the joint.

17. A learning apparatus comprising:
at least one processor,
wherein the processor is configured to:
construct a segmentation model that segments a region of one or more objects including a target object included in an input image, by performing machine learning using a plurality of sets of pseudo images and pseudo mask images generated by the image generation apparatus according to claim 1 as training data.

18. The learning apparatus according to claim 17,
wherein the processor is configured to:
construct the segmentation model by performing machine learning using a plurality of sets of original images and mask images as training data.

19. A segmentation model constructed by the learning apparatus according to claim 17.

20. An image processing apparatus comprising:
at least one processor,
wherein the processor is configured to derive a mask image in which one or more objects included in a target image to be processed are masked, by segmenting a region of one or more objects including a target object included in the target image using the segmentation model according to claim 19.

21. The image processing apparatus according to claim 20,
wherein the processor is configured to discriminate a class of the target object masked in the mask image using a discrimination model that discriminates a class of a target object included in a mask image.

22. An image generation method comprising:
acquiring an original image and a mask image in which masks are applied to one or more regions respectively representing one or more objects including a target object in the original image;
deriving a pseudo mask image by processing the mask in the mask image; and
deriving a pseudo image that has a region based on a mask included in the pseudo mask image and has the same representation format as the original image, based on the original image and the pseudo mask image.

23. A learning method of constructing a segmentation model that segments a region of one or more objects including a target object included in an input image, by performing machine learning using a plurality of sets of pseudo images and pseudo mask images generated by the image generation method according to claim 22 as training data.

24. An image processing method comprising:
deriving a mask image in which one or more objects included in a target image to be processed are masked, by segmenting a region of one or more objects including a target object included in the target image using the segmentation model according to claim 19.

25. A non-transitory computer-readable storage medium that stores an image generation program causing a computer to execute:
a procedure of acquiring an original image and a mask image in which masks are applied to one or more regions respectively representing one or more objects including a target object in the original image;
a procedure of deriving a pseudo mask image by processing the mask in the mask image; and
a procedure of deriving a pseudo image that has a region based on a mask included in the pseudo mask image and has the same representation format as the original image, based on the original image and the pseudo mask image.

26. A non-transitory computer-readable storage medium that stores a learning program causing a computer to execute:
a procedure of constructing a segmentation model that segments a region of one or more objects including a target object included in an input image, by performing machine learning using a plurality of sets of pseudo images and pseudo mask images generated by the image generation method according to claim 22 as training data.

27. A non-transitory computer-readable storage medium that stores an image processing program causing a computer to execute:
a procedure of deriving a mask image in which one or more objects included in a target image to be processed are masked, by segmenting a region of one or more objects including a target object included in the target image using the segmentation model according to claim 19.
